(19) United States
(12) Patent Application Publication
(10) Pub. No.: US 2025/0118414 A1
(43) Pub. Date: Apr. 10, 2025

(54) Method and Device for Planning an MR Examination on an MRT System

(71) Applicant: Siemens Healthineers AG, Forchheim (DE)

(72) Inventors: Mario Zeller, Erlangen (DE); Dominik Paul, Bubenreuth (DE)

(73) Assignee: Siemens Healthineers AG, Forchheim (DE)

(21) Appl. No.: 18/910,160

(22) Filed: Oct. 9, 2024

(30) Foreign Application Priority Data
Oct. 9, 2023 (DE) 10 2023 209 818.0

(51) Int. Cl. G16H 20/40; A61B 5/00; A61B 5/055

(52) U.S. Cl. CPC G16H 20/40; A61B 5/0077; A61B 5/055

(57) ABSTRACT

A method for planning an MR examination on an MRT system may include: provision of geometric information about an examination area, provision of quality information, comprising information about a homogeneity of a main magnetic field and/or of gradient fields in the examination area and/or information about a distortion correction of corrected images of the examination area, specification of a number of homogeneity areas in the examination area, based on a determination of whether values of the quality information lie in a specified value range, determination of position data based on location and shape of the number of homogeneity areas and of the examination area, and output of the position data.

### CROSS REFERENCE TO RELATED APPLICATIONS

This patent application claims priority to German Patent Application No. 10 2023 209 818.0, filed Oct. 9, 2023, which is incorporated herein by reference in its entirety.

### BACKGROUND

### Field

The disclosure relates to a method and a device for planning a magnetic resonance (MR) examination on a magnetic resonance tomography (MRT) system, and an MRT system.

### Related Art

MRT is an established examination method in medicine. In a strong, homogenous magnetic field (also referred to as “B0” or “constant magnetic field”), which is overlaid by gradient fields, materials are magnetically excited in the body and their signals are measured.

Some MR systems have a restricted homogeneity of the constant magnetic field or a restricted linearity of the gradient fields. The result is that the area that can meaningfully be used for imaging is restricted compared to expensive systems.

A visible effect of the lower quality of the magnetic fields is that some acquired areas in the final image are no longer fully contained due to effects of the distortion correction. They are basically cut off. Furthermore, there may be restrictions on fat saturation during scans. For these reasons it may then be the case that for large patients a region of the body (for example the thorax) cannot be captured in full.

Document US 2022/0397623 A1 describes a method which supports the examination personnel during planning on localizer images, in order to prevent regions being cut off by the distortion correction. However, this cannot compensate for unfavorable patient positioning during preparation for the scan.

Document U.S. Pat. No. 11,540,741 B2 described a method for supporting the operating personnel during coil positioning and selection using light projection and 3D camera.

### BRIEF DESCRIPTION OF THE DRAWINGS/FIGURES

The accompanying drawings, which are incorporated herein and form a part of the specification, illustrate the embodiments of the present disclosure and, together with the description, further serve to explain the principles of the embodiments and to enable a person skilled in the pertinent art to make and use the embodiments.

FIG. 1 a schematic representation of a magnetic resonance tomography system according to an exemplary embodiment of the disclosure.

FIG. 2 a flow chart for a possible operational sequence of an inventive method according to an exemplary embodiment of the disclosure.

FIG. 3 a possible output of the inventive method according to an exemplary embodiment of the disclosure.

FIG. 4 a further possible output of the inventive method according to an exemplary embodiment of the disclosure.

The exemplary embodiments of the present disclosure will be described with reference to the accompanying drawings. Elements, features and components that are identical, functionally identical and have the same effect are—insofar as is not stated otherwise-respectively provided with the same reference character.

### DETAILED DESCRIPTION

In the following description, numerous specific details are set forth in order to provide a thorough understanding of the embodiments of the present disclosure. However, it will be apparent to those skilled in the art that the embodiments, including structures, systems, and methods, may be practiced without these specific details. The description and representation herein are the common means used by those experienced or skilled in the art to most effectively convey the substance of their work to others skilled in the art. In other instances, well-known methods, procedures, components, and circuitry have not been described in detail to avoid unnecessarily obscuring embodiments of the disclosure. The connections shown in the figures between functional units or other elements can also be implemented as indirect connections, wherein a connection can be wireless or wired. Functional units can be implemented as hardware, software or a combination of hardware and software.

An object of the present disclosure is to specify a method and device for planning an MR examination on an MRT system, and an MRT system with which the above-described disadvantages can be obviated.

An inventive method may be used for planning an MR examination on an MRT system, i.e. in particular for checking whether an object to be examined is appropriately positioned or whether an examination area has been appropriately selected. It may comprise the following steps:
provision of geometric information about an examination area,
provision of quality information, comprising information about a homogeneity of a main magnetic field and/or of gradient fields in the examination area and/or information about a distortion correction of corrected images of the examination area,
specification of a number of homogeneity areas in the examination area, based on a determination of whether values of the quality information lie in an specified value range,
determination of position data based on location and shape of the number of homogeneity areas and of the examination area, and output of the position data.

The geometric information about the examination area is information that specifies the position and size of the examination area and in particular also its shape. This can be done for example by specifying a body region to be examined or by arranging the examination area in a virtual user interface. The examination area can be an area of the patient couch and an object on this area is regarded as the object to be examined, but the examination area may be an area of the object, in particular a three-dimensional area.

The geometric information about the examination area can be provided by an operative, in particular by specifying the body region to be examined, e.g. “Left Foot.”

It should be noted here that the examination area can generally be shaped and positioned entirely in line with the wishes of an operative and may well extend into areas where the homogeneity of constant magnetic field or gradient fields is poor, as soon as the patient table has been moved into the patient tunnel.

The quality information may comprise information which provides an insight into the expected quality of the images. The quality may be influenced by the local quality of the magnetic fields and if appropriate by the reconstruction used. The quality information to this end may comprise information:
about a homogeneity of a main magnetic field (at least) in the examination area and/or
about a homogeneity of gradient fields (at least) in the examination area and/or
information about a distortion correction of corrected images of the examination area.

This distortion correction in this case also reflects the homogeneity of the magnetic fields, since the distortion depends on local inhomogeneities.

The quality information is basically known and specific to the MRT system used. It can for example be stored in a dataset on a data memory of the MRT system and can be provided as required. However, it can also be measured, for example by measurements of the magnetic field.

If the quality information is provided, a homogeneity area in the examination area can be specified. This is done on the basis of a determination of whether values of the quality information lie in a specified value range. A limit value can for example be specified beforehand for the homogeneity of the constant magnetic field and can be checked to see what this homogeneity looks like in the examination area. Each position at which the homogeneity is above the limit value is assigned to the homogeneity area; if the homogeneity is less than this, then not. However, multiple homogeneity areas can also be specified. This may be done by specifying multiple limit values and a first homogeneity area may comprise areas with the best homogeneity and further homogeneity areas comprise areas with lower homogeneity (from different value ranges in each case with decreasing homogeneity).

It should be noted here that, in a very simple form of embodiment, homogeneity areas can be two-dimensional and can relate for example to the patient couch. However, since the magnetic fields and the objects to be examined are three-dimensional, the homogeneity areas may also be three-dimensional. In an exemplary embodiment, a homogeneity area may extend at least over the entire volume of the object to be examined in the examination area. The volume can for example be determined by 3D camera images or by a model of the object, and its position estimated.

The position data may be based on the location and shape of the number of homogeneity areas and of the examination area. Thus, in a simple example it can comprise the position and size of rectangles on the examination table, but it may comprise the position and shape of three-dimensional volumes. However, the position data can also comprise data from a comparison of the position and shape of the number of homogeneity areas with the examination area, for example whether a foot is optimally positioned in respect of a homogeneity area.

Position data can be output in various ways, such as in the form of a display of a representation of the number of homogeneity areas or in the form of control data.

A homogeneity area (or the representation thereof) can be displayed by a projection directly onto the patient couch or it can be displayed on a screen, wherein an image of the examination area may be overlaid with an image of the homogeneity area. However, alternatively or additionally, a result of a comparison of a homogeneity area with the examination area can be output, i.e. for example a warning that parts of the examination area or of the object to be examined lie outside the homogeneity area.

An output of control data can be used to move the patient couch automatically into another position. The control data may be sent to a unit used for positioning the patient couch or to a screen where an operative can read off the optimal positioning of the patient couch. Thus, it is possible to move the patient couch (if appropriate also automatically) so that the examination area (here this means the part of an object to be examined or a body region to be examined) is optimally aligned as regards the number of homogeneity areas. The expression “optimally aligned” here means that a maximum volume (or a maximum surface) of the examination area is positioned inside the homogeneity area with the best quality (the best values as regards the quality information, in particular the best homogeneity).

An inventive device may be used for planning an MR examination on an MRT system. It may comprise the following components:
a data interface configured to receive geometric information about an examination area,
a quality unit configured to retrieve quality information, comprising information about a homogeneity of a main magnetic field and/or of gradient fields in the examination area and/or information about a distortion correction of corrected images of the examination area,
a homogeneity unit configured to specify a number of homogeneity areas in the examination area, based on a determination of whether values of the quality information lie in a specified value range,
a position unit configured to determine position data based on location and shape of the number of homogeneity areas and of the examination area and to output the position data.

The device may be configured to execute an inventive method. The function of the components of the device has already been described in connection with the method.

An inventive MRT system may comprise an inventive device and/or may be configured to perform an inventive method.

The disclosure can in particular be realized in the form of a computer unit with appropriate software. To this end, the computer unit can for example have one or more interworking microprocessors or the like. In particular, it can be realized in the form of appropriate software program parts in the computer unit. A largely software-based realization has the advantage that even computer units already in use can easily be retrofitted by a software or firmware update, in order to work in the inventive manner. In this respect, the object is also achieved by a corresponding computer program product with a computer program which can be loaded directly into a memory device of a computer unit, with program sections in order to execute all steps of the inventive method when the program is executed in the computer unit. In addition to the computer program, such a computer program product can if appropriate comprise additional elements, such as for example documentation and/or additional components, including hardware components such as for example hardware keys (dongles, etc.) for use of the software.

For transport to the computer unit and/or for storage on or in the computer unit, a computer-readable medium, for example a memory stick, hard disk or other transportable or permanently installed data carrier, can be used, on which are stored the program sections of the computer program that can be read and executed by a computer unit.

Further particularly advantageous embodiments and developments of the disclosure emerge from the dependent claims and the following description, wherein the claims in one claim category can also be developed analogously to the claims and description parts for another claim category and in particular individual features of different exemplary embodiments or variants can also be combined to form new exemplary embodiments or variants.

In an exemplary embodiment, the examination area is specified by retrieving information about the examination, thus for example specifying which body part is to be examined and automatically determining where this body part typically lies in a specified standard location of a patient. The accuracy can in this case be further improved by using camera images of the patient.

Alternatively, or additionally, the examination area may be specified by positioning an MRT coil on a patient. In particular, in this case the position of the coil on the patient couch may be determined and the acquisition area of the coil or of the area covered by the coil may be specified as the examination area.

Alternatively, or additionally, the examination area may be specified by a selection by an operative. An operative may specify the position and size and in particular also the shape of the examination area, for example by means of a graphical user interface.

The specification may be done on a touchscreen, by a pressure bar on the patient couch or by gesture control.

The geometric information about the position and size and in particular also the shape of the examination area is made available to the method.

In an exemplary embodiment, a method may (e.g., additionally) comprise:
acquisition of a number of optical images of the examination area with an object to be examined with a camera,
capture of geometric information about the spatial location and the volume of an object to be examined (e.g. of a patient) in the examination area based on the acquired images, wherein the determination of position data based on location and shape of the number of homogeneity areas and of the examination area and output of the position data, thus for example the display of the number of homogeneity areas at the position of the examination area and/or of a result of a comparison of the number of homogeneity areas with the examination area or positioning of an object, takes into consideration the geometric information about the object.

The location of the object to be examined can advantageously be captured in three-dimensional space and the homogeneity in this space can be considered.

The camera provides optical images of the examination area with an object to be examined. If a 3D camera was used, the volume of the object or the 3D shape thereof can be estimated directly. If a 2D camera was used, the volume of the object can for example be determined from a model.

The geometric information may comprise at least the spatial location and the volume of the object to be examined in the examination area, thus for example a foot, based on the acquired images.

The number of homogeneity areas at the position of the examination area and/or a result of a comparison of the number of homogeneity areas with the examination area is then displayed, taking into consideration the geometric information about the object. Thus, it is in particular determined whether the object lies in a homogeneity area or extends beyond it. Then for example the object together with the homogeneity area can be shown on a display in a 3D representation or from multiple perspectives. It is however also possible to determine whether the object (e.g. a body area) lies entirely within the homogeneity area. If not, i.e. if something extends out from the object over the homogeneity area, information to this effect can be output.

Accordingly, an output of control data can take into consideration the geometric information about the object. Thus, here too in particular it is determined whether the object lies in a homogeneity area or extends beyond it. The optimal position of the object can then be determined and the object can correspondingly be automatically positioned or data about its optimal position can be displayed.

At least one homogeneity area is in this case a three-dimensional area and the geometric information is likewise three-dimensional information about the object to be examined. Thus, the above-described 3D examination is possible. The geometric information may be determined from optical images from a 3D camera or from images from a 2D camera using a three-dimensional model of the object.

In an exemplary embodiment, a device may comprise:
a camera, in particular a 3D camera, for the acquisition of optical images of the examination area,
a geometry unit configured to receive optical images of the examination area, and to capture geometric information about the spatial location and the volume of an object to be examined (for example a patient) in the examination area based on the acquired images.

The device may be configured so that the determination of position data based on location and shape of the number of homogeneity areas and of the examination area and output of the position data, in particular the display of the number of homogeneity areas at the position of the examination area and/or of a result of a comparison of the number of homogeneity areas with the examination area or a positioning of an object, takes into consideration the geometric information about the object.

The examination area may be a body region of a person and the geometric information may comprise information about the spatial location of the body region, information about the volume of the body region and/or the height profile of the body region. The homogeneity area may be defined not only by a surface, but by a volume. Additionally, or alternatively, for the determination of the examination area or for the volume of a body region, information about patient size and weight could be provided, for example by manually inputting this information or from an RIS/HIS (Radiology/Hospital Information System). As well, (image-based) information from preliminary examinations can be used for this.

The quality information may comprise information about a spatially resolved homogeneity of a main magnetic field of the MRT system in the examination area. In this case to specify a first homogeneity area it is determined whether the homogeneity at a position exceeds a predefined first limit value. To specify a second homogeneity area, it may be determined whether the homogeneity at a position exceeds a predefined second limit value, but in particular lies below the first limit value.

Alternatively, or additionally, the quality information may comprise information about a spatially resolved homogeneity of a gradient field of the MRT system in the examination area. In this case, to specify a first homogeneity area it is determined whether the homogeneity at a position exceeds a predefined first limit value. To determine a second homogeneity area, it may be determined whether the homogeneity at a position exceeds a predefined second limit value, but in particular lies below the first limit value.

Alternatively, or additionally, the quality information may comprise information about spatially resolved distortions in subsequently reconstructed images of the examination area. In this case, to specify a first homogeneity area it is determined whether a distortion at an image position exceeds a predefined first limit value. To determine a second homogeneity area, it may be determined whether the distortion at an image position exceeds a predefined second limit value, but in particular lies below the first limit value.

Alternatively, or additionally, the quality information may comprise information about a spatially resolved quality of the image reconstruction in subsequently reconstructed images of the examination area. In this case, to specify a first homogeneity area it is determined whether a quality at an image position exceeds a predefined first limit value. To specify a second homogeneity area, it may be determined whether the quality at an image position exceeds a predefined second limit value, but in particular lies below the first limit value.

As part of the display, the homogeneity area may be projected onto the examination area or may be displayed on a display. Suitable projection apparatuses are known. The homogeneity area can for example be projected as a light field onto a person on the patient couch. It is also possible that an image of the object to be acquired is shown on a display together with the homogeneity area.

When displaying the homogeneity area, a plurality of different homogeneity areas are displayed in different ways, in particular in different colors. For example, the homogeneity area with the best homogeneity can be displayed in a first color, e.g. green, and other homogeneity areas with poorer homogeneity can be displayed in other colors, e.g. yellow and/or red. However, a central homogeneity area can also be displayed with a number of surrounding homogeneity areas, e.g. as a light field with dark boundaries or as a pattern of bright boundaries. In addition, a notice can also be displayed, for example that the body region to be examined (e.g. the left foot) does not lie completely within the homogeneity area.

A method according to an exemplary embodiment may comprise: comparing the examination area with the homogeneity area.

If the comparison finds that the examination area is larger than the homogeneity area, information is output (for example to the operating personnel). This information may comprise warning information, which is in particular output by displaying a warning notice, for example as a projection onto the examination area or as a representation on a display, and/or by outputting an acoustic signal. Alternatively, or additionally, this information may comprise positioning information which specifies where the examination area should be positioned in order to lie in the homogeneity area. This allows the operating personnel to change the patient positioning appropriately prior to the start of the examination. The positioning information can for example suggest a shift on the table or if appropriate that an extremity (the chest, etc.) should be positioned higher or lower.

If the comparison finds that the examination area is larger than the homogeneity area, then, based on the homogeneity area, the examination area may be automatically subdivided into a plurality of examination areas and the method is performed once again for each of these subdivided examination areas. Thus, a number of homogeneity areas are output for each (partial) examination area, wherein each (partial) examination area can be positioned suitably for the examination. Alternatively, or additionally, a positioning of each of these subdivided examination areas in the homogeneity area can be determined. A measuring protocol may be extended automatically to the plurality of examination areas, in particular by the duplication of the measurement steps and specification of the respective centering position. The split into multiple examination areas would also result in two or more acquisitions, since each (partial) examination area would be acquired separately. As a result, the examination time is extended accordingly. Hence, information about effects on the measurement time may also be output.

For this, a device may comprise a comparison unit configured to compare the examination area with the homogeneity area. If the comparison finds that the examination area is larger than the homogeneity area, information may be output, for example to the operating personnel. The comparison unit may be configured, if it is found that the examination area is larger than the homogeneity area, on the basis of the homogeneity area, automatically to subdivide the examination area into a plurality of examination areas and to determine a positioning of each of these subdivided examination areas in the homogeneity area.

The MR examination may take place as part of an interventional imaging. The inventive method offers great advantages here, since in these examinations a particular geometric fidelity is in particular important.

The quality unit may comprise a database in which the quality information is contained. This is possible, since the geometry of the magnetic coils is known and the magnetic field can as a result be calculated. It is also possible to measure the magnetic field beforehand and to save the measured values (preferably relating directly to the homogeneity) as a dataset. Alternatively, or additionally, the quality unit may comprise sensors for measuring the homogeneity of the main magnetic field and/or of a gradient field of the MRT system.

AI-based methods (AI: “Artificial Intelligence”) may be used for the inventive method. An artificial intelligence is based on the principle of machine-based learning and is generally performed with an algorithm capable of learning that has been correspondingly trained. The expression “machine learning” is frequently used for machine-based learning, this also including the principle of “deep learning.”

Components of the disclosure may be configured as a “cloud service.” Such a cloud service is used to process data, in particular by means of an artificial intelligence, but can also be a service based on conventional algorithms or a service in which an evaluation by humans takes place in the background. In general, a cloud service (also referred to as “cloud” for short below) is an IT infrastructure in which for example memory space or computing power and/or application software is made available via a network. The communication between the user and the cloud in this case takes place by means of data interfaces and/or data transfer protocols. In the present case, the cloud service may make both computing power and application software available.

As part of a method according to the disclosure, data obtained in connection with the disclosure may be provided to the cloud service via the network. This may comprise a computing system which in general does not include the user's local computer. The method can in this case be realized by means of a command constellation in a network. The data calculated in the cloud is subsequently resent to the user's local computer via the network.

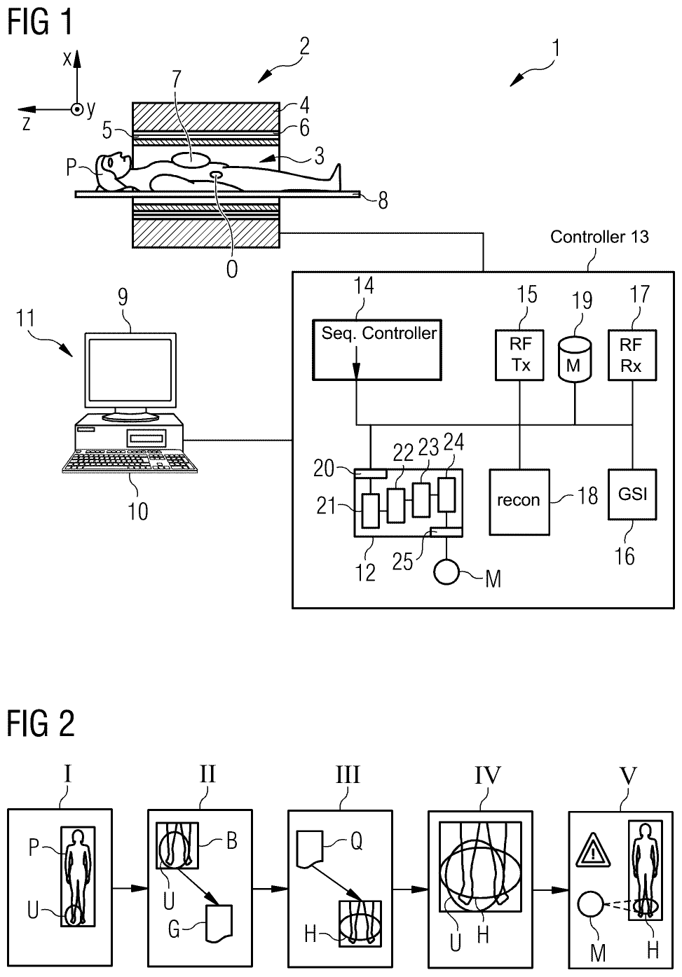

FIG. 1 roughly schematically shows a magnetic resonance tomography system 1. It firstly may comprise the actual magnetic resonance scanner 2 with an examination space 3 or patient tunnel, in which a patient or test subject, in whose body the examination object O itself is located, is positioned on a couch 8.

The magnetic resonance scanner 2 is normally equipped with a constant field magnet system 4, a gradient system 6 as well as an RF transmission antenna system 5 and an RF receiving antenna system 7. In the exemplary embodiment shown the RF transmission antenna system 5 is a whole-body coil permanently installed in the magnetic resonance scanner 2, whereas the RF receiving antenna system 7 consists of local coils to be arranged on the patient or test subject (symbolized here only by a single local coil). However, in principle the whole-body coil can also be used as an RF receiving antenna system and the local coils as an RF transmission antenna system, providing these coils can each be switched to different operating modes. The constant field magnet system 4 is here normally configured so that it generates a constant magnetic field in the longitudinal direction of the patient, i.e. along the longitudinal axis, running in the z-direction, of the magnetic resonance scanner 2. The gradient system 6 normally may comprise individually controllable gradient coils, in order to be able to switch gradients in the x-, y- or z-direction independently of one another. Furthermore, the magnetic resonance scanner 2 contains shim coils (not shown), which can be configured in the normal manner.

The magnetic resonance tomography system shown here is a whole-body system with a patient tunnel, into which a patient can be introduced completely. However, in principle the disclosure can also be used on other magnetic resonance tomography systems, e.g. with a laterally open, C-shaped housing. The only important thing is that corresponding acquisitions of the examination object O can be produced.

The magnetic resonance tomography system 1 further has a central controller 13 which is used to control the MR system 1. This central controller 13 may comprise a sequence controller 14. This is used to control the operational sequence of radio-frequency pulses (RF pulses) and of gradient pulses as a function of a selected pulse sequence or an operational sequence of multiple pulse sequences for the acquisition of multiple slices in a volume area of interest of the examination object within a scanning session. Such a pulse sequence can for example be predefined and parameterized within a measuring or control protocol. Normally different control protocols for different measurements or scanning sessions are stored in a memory 19 and can be selected (and if necessary modified) by an operator and then used to perform the scan.

The examination area can be specified on the basis of selected pulse sequences or on the basis of the positioning of the above-mentioned RF receiving antenna system 7.

To output the individual RF pulses in a pulse sequence the central controller 13 has a radio-frequency transmission device (transmitter) 15 which generates and amplifies the RF pulses and feeds them to the RF transmission antenna system 5 via a suitable interface (not shown in detail). To control the gradient coils of the gradient system 6, the controller 13 has a gradient system interface 16 in order to switch the gradient pulses appropriately in accordance with the predefined pulse sequence. The diffusion gradient pulses and spoiler gradient pulses could be applied via this gradient system interface 16. The sequence controller 14 communicates in an appropriate manner, for example by emitting sequence control data, with the radio-frequency transmission device 15 and the gradient system interface 16 for the execution of the pulse sequence.

The controller 13 additionally has a radio-frequency receiving device (receiver) 17 (likewise communicating in an appropriate manner with the sequence controller 14), in order to receive magnetic resonance signals within the readout window predefined by the pulse sequence in a coordinated manner by means of the RF receiving antenna system 7 and thus to acquire the raw data.

A reconstruction unit (reconstructor, reconstructing processor) 18 may be configured to reconstruct magnetic resonance image data based on the acquired raw data. This reconstruction also generally takes place on the basis of parameters which can be predefined in the respective measuring or control protocol. This image data can then for example be stored in a memory 19.

The person skilled in the art in principle knows how appropriate raw data can be acquired in detail by an irradiation of RF pulses and switching of gradient pulses and how MR images or parameter maps can be reconstructed from them, and this is hence not explained here in greater detail.

The controller 13 further has an inventive device (planning processor) 12 configured to plan an MR examination on this MRT system 1. In an exemplary embodiment, the device 12 includes processing circuitry that is configured to perform one or more operations and/or functions of the device 12, such as to plan the MR examination. Additionally, or alternatively, one or more components (e.g., 20, 21, 22, 23, 24, 25) of the device 12 may include processing circuitry that is configured to perform one or more respective operations and/or functions of the component(s).

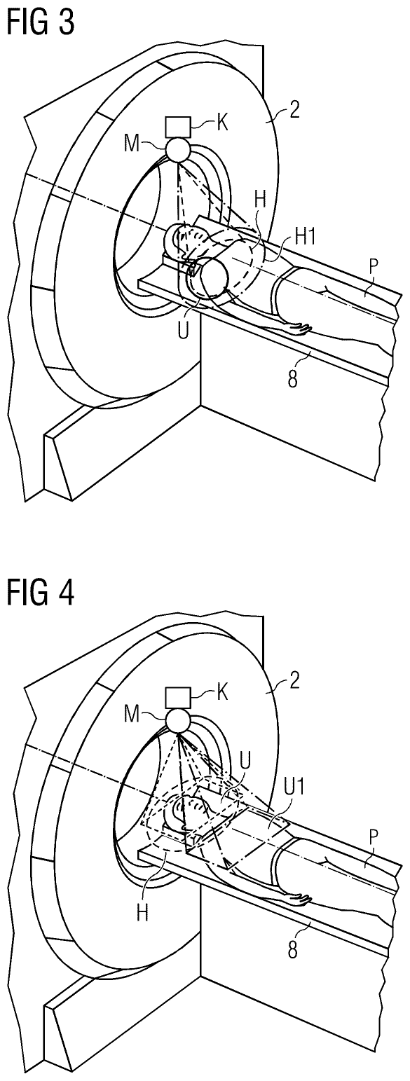

This device 12 may comprise the following components:
A data interface 20 configured to receive geometric information G about an examination area U. This information can for example be input by an operative.
A camera K (not shown, see FIGS. 3 and 4), in particular a 3D camera K, for acquiring optical images B of the examination area U.
A geometry unit 21 configured to receive the optical images B from the camera K, and to capture geometric information G about the spatial location and the volume of an object to be examined in the examination area U based on the acquired images B.
A quality unit 22 configured to retrieve quality information Q, for example from the memory 19, comprising information about a homogeneity of the main magnetic field and/or the gradient fields in the examination area U and/or information about a distortion correction of corrected images B of the examination area U.
A homogeneity unit 23 configured to specify a number of homogeneity areas H, H1 in the examination area U, based on a determination of whether values of the quality information Q lie in a specified value range.
A position unit 24 configured to output information for the determination of position data based on location and shape of the number of homogeneity areas H, H1 and of the examination area U and to output the position data, in particular for the display of the number of homogeneity areas H, H1 at the position of the examination area U and/or of a result of a comparison of the number of homogeneity areas H, H1 with the examination area U and/or a positioning of the object O, such as taking into consideration the geometric information G about the object O.
A comparison unit (comparator) 25 configured to compare the examination area U with the homogeneity area H, H1, wherein if the comparison finds that the examination area U is larger than the homogeneity area H, H1, information is output. The comparison unit 25 may be also configured, if it is found that the examination area U is larger than the homogeneity area H, H1, based on the homogeneity area H, H1 to subdivide the examination area U automatically into a plurality of examination areas U, U1 and also to determine a positioning of each of these subdivided examination areas U in the homogeneity area H, H1.

The central controller 13 can be operated via a terminal 11 with an input interface 10 and an output interface (e.g., display unit) 9, via which thus the whole magnetic resonance tomography system 1 can also be operated by an operative. Magnetic resonance tomography images can also be displayed on the output interface 9, and by means of the input interface 10, if appropriate in combination with the output interface 9, scans can be planned and started and in particular control protocols selected and if appropriate modified.

The inventive magnetic resonance tomography system 1 and in particular the controller 13 can additionally have a plurality of further components, not shown here individually but normally present in such systems, such as for example a network interface, in order to connect the whole system to a network and to be able to exchange raw data and/or image data or parameter maps, as well as further data, such as for example patient-related data or control protocols.

How appropriate raw data can be acquired by irradiating RF pulses and generating gradient fields and how magnetic resonance tomography images can be reconstructed therefrom is in principle known to the person skilled in the art and is not explained in detail here.

FIG. 2 shows a flow chart for a possible operational sequence of an inventive method for planning an MR examination on an MRT system 1, as shown for example in FIG. 1.

In step I geometric information G about an examination area U is provided, for example by an operative at an input terminal.

In step II a number of images B of the examination area U with an object to be examined O are acquired with a camera K and from these images B geometric information G about the spatial location and the volume of an object to be examined O in the examination area U are captured based on the acquired images B.

In step III quality information Q, comprising information about a homogeneity of a main magnetic field and/or of gradient fields in the examination area U or information about a distortion correction of corrected images B of the examination area U, is provided and, based on a determination of whether values of the quality information Q lie in a specified value range, a homogeneity area H in the examination area U is specified.

In step IV the homogeneity area H at the position of the examination area U is displayed. Alternatively, a result of a comparison of the homogeneity area H with the examination area U could also be displayed, thus for example whether the foot displayed here lies in the homogeneity area H, which in this example it does not.

FIG. 3 shows a possible output of the inventive method (see FIG. 2). For example, the quality information Q here contains information about a spatially resolved homogeneity of a main magnetic field of the MRT system in the examination area U. To specify a first homogeneity area H it was then determined whether the homogeneity at a position exceeds a predefined first limit value. In addition, a second homogeneity area H1 was specified, in which it was determined whether the homogeneity at a position exceeds a predefined second limit value, but lies below the first limit value.

Both the homogeneity areas H, H1 are then projected onto the examination area U, as can be seen in the figure, wherein the first (inner) homogeneity area H is for example displayed in green (shown here as a dashed line) and the second (outer) homogeneity area H1 is for example displayed in yellow (shown here as a dot-dashed line).

FIG. 4 shows a further possible output of the inventive method. A comparison was first made here of the examination area U with the homogeneity area H and in the comparison it was found that the examination area U (which initially corresponded to the common surface of both the examination areas U1, U2 shown) was larger than the homogeneity area H.

The original examination area U was then automatically subdivided into two examination areas U, U1 and the method was performed once again for each of these subdivided examination areas U, U1. These two examination areas U, U1 can now be acquired one after the other, which prolongs the examination time but leads to much better results.

In conclusion it is once again noted that the disclosure described above in detail relates solely to exemplary embodiments that can be modified by the person skilled in the art in a variety of ways, without departing from the scope of the disclosure. Further, the use of the indefinite article “a” or “an” does not rule out that the features in question may also be present multiple times. Likewise, the term “unit” does not rule out that the components in question consist of multiple interacting subcomponents which if appropriate may also be distributed spatially. The term “a number” is to be read as “at least one.”

To enable those skilled in the art to better understand the solution of the present disclosure, the technical solution in the embodiments of the present disclosure is described clearly and completely below in conjunction with the drawings in the embodiments of the present disclosure. Obviously, the embodiments described are only some, not all, of the embodiments of the present disclosure. All other embodiments obtained by those skilled in the art on the basis of the embodiments in the present disclosure without any creative effort should fall within the scope of protection of the present disclosure.

It should be noted that the terms “first”, “second”, etc. in the description, claims and abovementioned drawings of the present disclosure are used to distinguish between similar objects, but not necessarily used to describe a specific order or sequence. It should be understood that data used in this way can be interchanged as appropriate so that the embodiments of the present disclosure described here can be implemented in an order other than those shown or described here. In addition, the terms “comprise” and “have” and any variants thereof are intended to cover non-exclusive inclusion. For example, a process, method, system, product or equipment comprising a series of steps or modules or units is not necessarily limited to those steps or modules or units which are clearly listed, but may comprise other steps or modules or units which are not clearly listed or are intrinsic to such processes, methods, products or equipment.

References in the specification to “one embodiment,” “an embodiment,” “an exemplary embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The exemplary embodiments described herein are provided for illustrative purposes, and are not limiting. Other exemplary embodiments are possible, and modifications may be made to the exemplary embodiments. Therefore, the specification is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computer). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact results from computing devices, processors, controllers, or other devices executing the firmware, software, routines, instructions, etc. Further, any of the implementation variations may be carried out by a general-purpose computer.

The various components described herein may be referred to as “modules,” “units,” or “devices.” Such components may be implemented via any suitable combination of hardware and/or software components as applicable and/or known to achieve their intended respective functionality. This may include mechanical and/or electrical components, processors, processing circuitry, or other suitable hardware components, in addition to or instead of those discussed herein. Such components may be configured to operate independently, or configured to execute instructions or computer programs that are stored on a suitable computer-readable medium. Regardless of the particular implementation, such modules, units, or devices, as applicable and relevant, may alternatively be referred to herein as “circuitry,” “controllers,” “processors,” or “processing circuitry,” or alternatively as noted herein.

For the purposes of this discussion, the term “processing circuitry” shall be understood to be circuit(s) or processor(s), or a combination thereof. A circuit includes an analog circuit, a digital circuit, data processing circuit, other structural electronic hardware, or a combination thereof. A processor includes a microprocessor, a digital signal processor (DSP), central processor (CPU), application-specific instruction set processor (ASIP), graphics and/or image processor, multi-core processor, or other hardware processor. The processor may be “hard-coded” with instructions to perform corresponding function(s) according to aspects described herein. Alternatively, the processor may access an internal and/or external memory to retrieve instructions stored in the memory, which when executed by the processor, perform the corresponding function(s) associated with the processor, and/or one or more functions and/or operations related to the operation of a component having the processor included therein.

In one or more of the exemplary embodiments described herein, the memory is any well-known volatile and/or non-volatile memory, including, for example, read-only memory (ROM), random access memory (RAM), flash memory, a magnetic storage media, an optical disc, erasable programmable read only memory (EPROM), and programmable read only memory (PROM). The memory can be non-removable, removable, or a combination of both.

1. A method for planning a magnetic resonance (MR) examination on a magnetic resonance tomography (MRT) system, the method comprising:
determining geometric information about an examination area;
determining quality information including: information corresponding to a homogeneity of a main magnetic field and/or of gradient fields in the examination area, and/or information about a distortion correction of corrected images of the examination area;
specifying a number of homogeneity areas in the examination area, based on a determination of whether values of the quality information lie in a specified value range;
determining position data based on location and shape of the number of homogeneity areas and of the examination area; and
outputting the position data in electronic form as a data file.

2. The method as claimed in claim 1, wherein the examination area is specified based on:
a positioning of an MRT coil on a patient;
a selection, by an operative, of the examination area, wherein the selection may be performed using a touchscreen, a pressure bar on the patient couch, or a gesture control; and/or
geometric information about the position and size of the examination area.

3. The method as claimed in claim 1, further comprising:
acquiring, using a camera, a number of optical images of the examination area with an object to be examined; and
capturing geometric information about the spatial location and the volume of the object to be examined in the examination area based on the acquired images.

4. The method as claimed in claim 3, wherein:
the determination of position data is based on the geometric information about the object, the outputting of the position data including displaying of the number of homogeneity areas at the position of the examination area and/or of a result of a comparison of the number of homogeneity areas with the examination area and/or a positioning of the object;
at least one homogeneity area is a three-dimensional area and the geometric information is three-dimensional (3D) information about the object to be examined determined from optical images from a 3D camera or from images from a two-dimensional (2D)-camera using a 3D model of the object; and/or
the examination area is a body region of a person and the geometric information comprises information about the spatial location of the body region, information about the volume of the body region and/or the height profile of the body region.

5. The method as claimed in claim 1, wherein the quality information comprises:
information about a spatially resolved homogeneity of a main magnetic field of the MRT system in the examination area, wherein, to specify a first homogeneity area, determining whether the homogeneity at a first position exceeds a predefined first limit value, and to specify a second homogeneity area, determining whether the homogeneity at a second position exceeds a predefined second limit value and is less than the first limit value;
a spatially resolved homogeneity of a gradient field of the MRT system in the examination area, wherein, to specify a first homogeneity area, determining whether the homogeneity at the first position exceeds the predefined first limit value, and to specify a second homogeneity area, determining whether the homogeneity at the second position exceeds a predefined second limit value and is less than the first limit value;
spatially resolved distortions in subsequently reconstructed images of the examination area, wherein, to specify the first homogeneity area, determining whether a distortion at an image position exceeds a predefined first limit value, and to specify a second homogeneity area, determining whether the distortion at the image position exceeds a predefined second limit value and is less than the first limit value; and/or
a spatially resolved quality of the image reconstruction in subsequently reconstructed images of the examination area, wherein, to specify a first homogeneity area, determining whether a quality at an image position exceeds a predefined first limit value, and to specify a second homogeneity area, determining whether the quality at an image position exceeds a predefined second limit value and is less than the first limit value.

6. The method as claimed claim 1, wherein outputting the position data comprises projecting the homogeneity area onto the examination area or displaying the homogeneity area on a display, a plurality of different homogeneity areas being differently projected or displayed in different colors and/or as a central homogeneity area with a number of surrounding homogeneity areas.

7. The method as claimed in claim 1, further comprising:
comparing the examination area with the homogeneity area, and output information in response to the comparison indicating that the examination area is larger than the homogeneity area,
wherein the outputted information comprises warning information, the warning information including a warning notice being displayed, an acoustic signal being output, and/or positioning information specifying where the examination area should be positioned in order to lie in the homogeneity area.

8. The method as claimed in claim 7, wherein, in response to the comparison indicating that the examination area is larger than the homogeneity area, the examination area is automatically subdivided, based on the homogeneity area, into a plurality of examination areas and the method is repeated for each of the subdivided examination areas and/or a positioning of each of the subdivided examination areas in the homogeneity area is determined, wherein a measuring protocol is extended automatically to the plurality of examination areas and/or information about effects on the measurement time is output.

9. The method as claimed in claim 1, wherein the MR examination takes place as part of an interventional imaging.

10. A computer program product embodied on a non-transitory computer-readable medium and including commands, which, when executed by a processor, cause the processor to perform the method of claim 1.

11. A non-transitory computer-readable storage medium comprising commands, when executed by a processor, causes the processor to perform the method of claim 1.

12. A device for planning a magnetic resonance (MR) examination on a magnetic resonance tomography (MRT) system, the device comprising:
a data interface configured to receive geometric information about an examination area;
a quality unit configured to retrieve quality information comprising: information about a homogeneity of a main magnetic field and/or of gradient fields in the examination area and/or information about a distortion correction of corrected images of the examination area;
a homogeneity unit configured to specify a number of homogeneity areas in the examination area, based on a determination of whether values of the quality information lie in a specified value range;
a position unit configured to determine position data based on location and shape of the number of homogeneity areas and of the examination area; and
an output interface configured to output the position data, including displaying the number of homogeneity areas at the position of the examination area and/or displaying a result of a comparison of the number of homogeneity areas with the examination area.

13. The device as claimed in claim 12, further comprising:
a camera configured to acquire optical images of the examination area;
a geometry unit configured to: receive optical images of the examination area; and capture geometric information about the spatial location and the volume of an object to be examined in the examination area based on the acquired images,
wherein the position unit is configured to determine the position data based on the geometric information, and the output interface is configured to display the number of homogeneity areas at the position of the examination area and/or display a result of a comparison of the number of homogeneity areas with the examination area and/or a positioning of the object.

14. The device as claimed in claim 12, wherein the quality unit comprises a database configured to store the quality information, and/or comprises sensors configured to measure the homogeneity of the main magnetic field and/or of a gradient field of the MRT system.

15. The device as claimed in claim 12, further comprising: a comparison unit configured to compare the examination area with the homogeneity area, wherein in response to the examination area being larger than the homogeneity area, information is output.

16. The device as claimed in claim 15, wherein the comparison unit is further configured to: in response to the examination area being larger than the homogeneity area, automatically subdivide the examination area, based on the homogeneity area, into a plurality of examination areas and determine a positioning of each of the subdivided examination areas in the homogeneity area.

17. An MRT system comprising the device as claimed in claim 12.

18. A device for planning a magnetic resonance (MR) examination on a magnetic resonance tomography (MRT) system, comprising:
one or more processors; and
memory storing instructions that, when executed by the one or more processors, configure the device to:
determine geometric information about an examination area;
determine quality information including: information corresponding to a homogeneity of a main magnetic field and/or of gradient fields in the examination area, and/or information about a distortion correction of corrected images of the examination area;
specify a number of homogeneity areas in the examination area, based on a determination of whether values of the quality information lie in a specified value range;
determine position data based on location and shape of the number of homogeneity areas and of the examination area; and
output the position data in electronic form as a data file.
